(19) United States
(12) Patent Application Publication
(10) Pub. No.: US 2012/0277576 A1
(43) Pub. Date: Nov. 1, 2012

(54) ECHOGENIC INFUSION PORT CATHETER

(76) Inventor: Chun Kee Lui, Apollo, PA (US)

(21) Appl. No.: 13/410,340

(22) Filed: Mar. 2, 2012

Related U.S. Application Data
(60) Provisional application No. 61/479,098, filed on Apr. 26, 2011.

(51) Int. Cl. A61B 8/00; B23P 17/04; A61B 17/34

(52) U.S. Cl. 600/424; 604/164.01; 604/164.13; 29/428

(57) ABSTRACT

Among other things, a delivery catheter assembly for insertion into a patient's vasculature is disclosed. In particular embodiments, the assembly includes a catheter, an obturator, and a hub. The hub is configured to maintain the proximal end of the catheter thereon. The hub and the proximal end of the obturator are configured to securely engage each other until a physician desires to release the obturator from the hub. When the catheter and obturator are assembled, the distal ends of the catheter and the obturator are adjacent or substantially aligned. In one form, the distal end of the obturator is made of an echogenic material that is visible with sonography or ultrasound imaging. In another form, a plurality of echogenic markers is positioned along the obturator including the distal end of the obturator. Beneficially, the position of the catheter is determined by locating the obturator using sonography or ultrasound imaging.

### REFERENCE TO RELATED APPLICATION

This application claims the benefit of U.S. Provisional Application No. 61/479,098, filed Apr. 26, 2011, which is hereby incorporated by reference.

### BACKGROUND

The present disclosure concerns an echogenic catheter that is highly visible using sonography and/or ultrasound imaging during placement of the echogenic catheter. Beneficially, fluoroscopy or X-rays are not necessary to determine the position of the echogenic catheter.

It is generally known that a catheter is a long thin hollow tube with two separate ends. A distal end of the catheter is to be inserted into the body of a medical patient. In one form, the opposite proximal end of the catheter remains outside the body and connects with a permanent hub. In another form, the opposite proximal end is designed to connect with an infusion port, and the assembled catheter and infusion port are implanted under the skin surface. Typically, catheters have one or more internal lumens with diameters that have a volume of sufficient size to allow for passage of wires, rods, liquids, gases, and other specially designed medical instruments.

Typically, when implanting a catheter, a guide wire is first advanced into the vasculature of the patient. Next, in one technique, the catheter is inserted over the guide wire. In another technique, first an introducer is inserted over the guide wire and then after placement of the introducer, the guide wire is removed from the patient and discarded. Next, the catheter is pushed through the introducer and into the patient. In either technique, fluoroscopy or X-rays are often used to aid in positioning the catheter. Accurate location and orientation of the catheter within the vasculature of the patient is very important since advancing the catheter too far may result in missing an intended site for delivery of medicament or implant or for obtaining a sample. Unwanted penetration of a blood vessel or other anatomical structure that lies beyond the intended location can also occur. Upon satisfactory positioning of the distal end of the catheter as determined with fluoroscopy or X-rays, the catheter can be used as the physician or other health professional intends. For example, the proximal end of the catheter may be trimmed so as to be relatively close to the patient's skin and then connected to a desired device, such as an infusion port for introducing fluids. As another example, the proximal end of the catheter is connected to a permanent hub, therefore, the proximal end does not need to be trimmed.

As can be appreciated, exposure to X-rays can be harmful to the patient. As such, catheterization that avoids X-rays altogether or reduces the dosage or number of exposures to such radiation reduces any risk of cancer or other damage that may be associated with such radiation for these medical patients. With current vascular catheterization depending on X-ray visualization, there is a need for improvement in this field.

### SUMMARY

This Summary is provided merely to introduce certain concepts and not to identify any key or essential features of the claimed subject matter.

In certain of its aspects, the present disclosure features embodiments of methods for positioning a catheter within the vasculature of a patient. In particular embodiments, such methods include providing a catheter with an echogenic obturator installed therein and a hub positioned in the catheter to retain the catheter. The catheter and the obturator each have a distal end opposite a proximal end wherein the distal ends of the catheter and the echogenic obturator are aligned. Moreover, the proximal end of the echogenic obturator is configured to releasably retain the hub. The catheter and the echogenic obturator are inserted into the vasculature of the patient. The position of the distal end of the catheter within the vasculature of the patient is determined with ultrasound imaging of the distal end of the echogenic obturator. The echogenic obturator is released from the hub after the determination of the position of the distal end of the catheter. In one embodiment, the proximal end of the echogenic obturator includes a cap configured to retain the hub. Additionally, this embodiment can include the cap having threads (e.g. an internal thread) and the hub having a thread (e.g. an external thread) configured to threadedly engage the thread(s) on the cap. In this embodiment, the echogenic obturator is rotated around the hub to unscrew the threads of the cap from the threads of the hub to release the obturator from the hub.

In other of its aspects, the present disclosure features embodiments of a delivery catheter including a catheter, an obturator, and a hub. In particular embodiments, the catheter and the obturator each have a distal end opposite a proximal end. The obturator is disposed at least partially within the catheter such that the distal ends of the catheter and the obturator are substantially aligned. Beneficially, the distal end of the obturator is made of an echogenic material that is visible with sonography or ultrasound imaging. As such, the distal end of the catheter can be located by imaging the distal end of the obturator. The hub has a proximal flange portion opposite a distal body portion wherein the distal body portion is configured to securely engage the catheter when the hub is assembled with the catheter. The proximal flange portion and the proximal end of the obturator are configured to connect with each other until a physician desires to disconnect the obturator from the hub and catheter. In one embodiment, the proximal end of the obturator includes a cap and each of the cap and the proximal hub portion are threaded so as to releasably connect with one another to retain the obturator on the hub. In another embodiment, the distal body portion includes a plurality of teeth configured to grip the interior of the proximal end of the catheter. In one form, the obturator includes a shaft that spans from the distal end to the proximal end and a plurality of markers made of echogenic material. Each of the plurality of markers is positioned at an interval, which intervals may be substantially uniform, along the shaft. In other embodiments, the catheter includes or is made of a radiopaque material to enable visualization of the catheter within the vasculature of the patient by fluoroscopy or X-rays after the obturator is removed.

In yet other of its aspects, the present disclosure features embodiments of methods of making a delivery catheter including providing a catheter and an obturator each having a distal end opposite a proximal end and a hub having a distal body portion opposite a proximal flange portion. The distal end of the obturator is made of an echogenic material. The distal body portion of the hub is inserted into the proximal end of the catheter to securely engage the catheter thereon. The distal end of the obturator is inserted into the proximal flange portion of the hub and the catheter until the distal end of obturator is aligned with the distal end of the catheter. Finally, the proximal end of the obturator is attached to the proximal flange portion of the hub.

Further forms, objects, features, aspects, benefits, advantages, and embodiments of the present disclosure will become apparent from a detailed description and drawings provided herewith.

### BRIEF DESCRIPTION OF THE DRAWINGS

FIG. 1 is a cross-sectional view of an echogenic catheter.

FIG. 2 is a cross-sectional view of an alternate embodiment of an echogenic catheter.

FIG. 3 is a cross-sectional view of another alternate embodiment of an echogenic catheter in a disassembled state.

FIG. 4 is a cross-sectional view of the FIG. 3 echogenic catheter in an assembled state.

### DESCRIPTION OF THE SELECTED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the claims is thereby intended. Any alterations and further modifications in the described embodiments, and any further applications of the principles of the disclosure as described herein are contemplated as would normally occur to one skilled in the art to which the disclosure relates. One embodiment is shown in great detail, although it will be apparent to those skilled in the relevant art that some features that are not relevant to the present disclosure may not be shown for the sake of clarity.

As disclosed above, in certain aspects, the present disclosure provides unique products and methods for positioning a delivery catheter within the vasculature of a patient. Embodiments of the delivery catheter assembly can include a catheter, an echogenic obturator, and an adapter or hub. In one embodiment the catheter assembly is assembled in a kit wherein the hub is installed in a proximal end of the catheter, and the echogenic obturator is attached to the hub and inserted through the hub and into the catheter. A distal end of the obturator may extend to or proximate to a distal end of the catheter. When the delivery catheter is assembled, the distal ends of the obturator and the catheter are next to or near one another such that the physician or other professional can determine the position of the catheter with sonography or ultrasound imaging of the echogenic obturator. The obturator is attached to the hub such that it can be easily removed from the hub without disturbing the connection between the catheter and hub, and/or without displacing the catheter tube after it has been inserted into the vasculature of the patient. With such easy removal of the obturator from the catheter not causing movement of the catheter, the final position of the catheter within the patient is not disturbed. Advantageously, the delivery catheter assembly can be positioned within the patient with the use of ultrasound imaging or sonography, rather than with X-rays or similar radiation.

After the catheter and obturator are properly positioned within the vasculature, the obturator can be detached from the hub and removed from the catheter. In one form, detaching the obturator from the hub includes rotating the obturator around the hub. With the obturator withdrawn from the catheter, the physician can use the catheter to perform the therapeutic, diagnostic or other tasks that are desired. For example, the physician may trim the proximal end of the catheter so that the amount of catheter extending from the patient is a manageable length. Such removal of the proximal portion of the catheter tube from the rest of the catheter within the body also serves to remove the hub from the rest of the tube. Alternatively, the physician may trim the proximal end of the catheter so that the trimmed end of catheter can be attached to an appropriate medical device and implanted under the skin surface. The physician may place an appropriate device, such as a connector for connecting a source of therapeutic material or a syringe or other device for obtaining a sample, an infusion port for selective introduction of medicaments, or other medical device to the end of the catheter created by trimming. As noted, X-rays may not be needed for placement of the catheter, but if the physician desires, the position of the catheter initially placed via ultrasound visualization may be verified with fluoroscopy or X-rays, prior to or after removal of the obturator from the catheter.

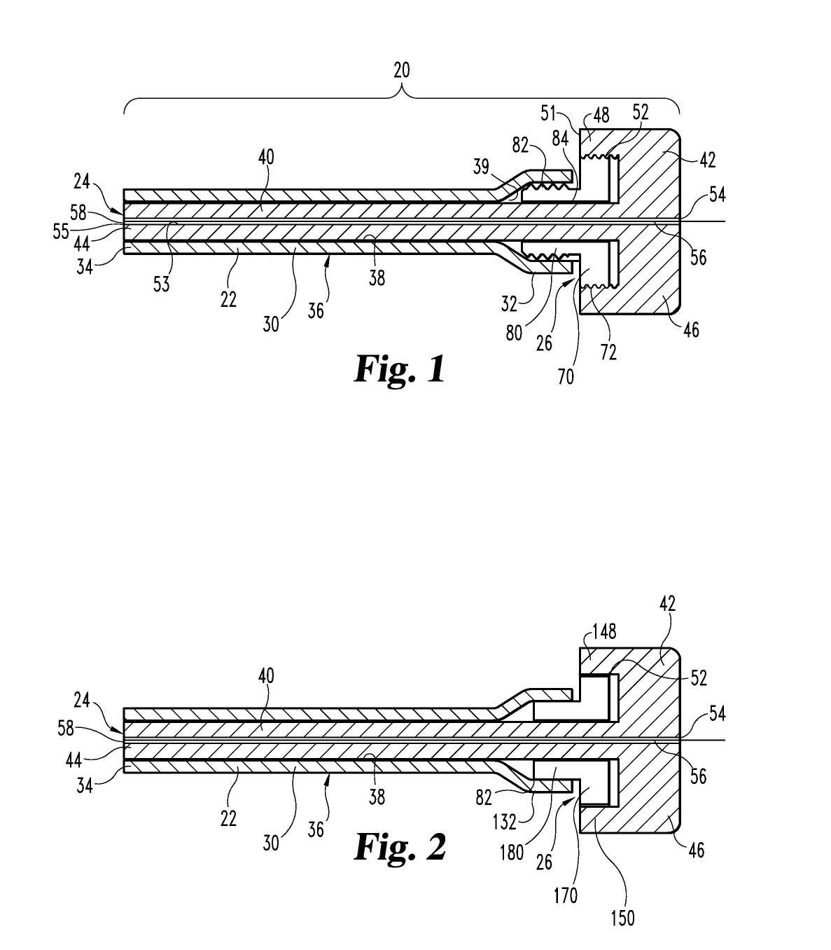

FIG. 1 illustrates an embodiment of a delivery catheter assembly or echogenic catheter assembly 20 of the present disclosure. Assembly 20 includes a hollow catheter 22, an echogenic obturator 24, and an adapter or hub 26. As illustrated and described in more detail below, the hub 26 is inserted in the catheter 22 to maintain or hold the catheter 22 thereon. Obturator 24 is inserted through hub 26 and attached to its outside. Catheter 22 and the obturator 24 are thus configured to connect with the hub 26 until the physician desires to remove the obturator 24 and/or hub 26.

Catheter 22 includes an elongate flexible body 30 having a proximal end 32 and an opposite distal end 34. In the illustrated embodiment, body 30 has a first diameter at distal end 34 that is substantially uniform throughout until body 30 approaches proximal end 32. A portion of proximal end 32 flares radially outwardly from the first diameter to a second diameter that is larger than the first diameter. The outwardly flaring portion forms a neck in this embodiment between two substantially constant-diameter portions, one being of a larger diameter than the other. The larger diameter of proximal end 32 permits body 30 to engage a distal portion 80 of the hub 26, as described in more detail below. In another embodiment, no neck is provided, with proximal end 32 being of the same substantially uniform diameter as distal end 34 and the rest of body 30. In either configuration, proximal end 32 forms an interference or press fit connection with the distal portion 80 of the hub 26 as described in more detail below. Generally, the physician is not required to apply a large amount of force to insert catheters into a patient. Therefore, the connection between the proximal end 32 of the catheter 22 and the hub 26 needs to be strong enough to simply maintain the firm connection between proximal end 32 and the distal portion 80 of hub 26 during the pushing and/or pulling of catheter 22 as it is maneuvered through the vasculature.

The hollow catheter 22 defines an outer surface 36 and an inner surface 38 surrounding a lumen 39. Lumen 39 has a diameter (i.e. the internal diameter of inner surface 38) sized to receive the obturator 24. In one form, the thickness of the catheter 22 from the inner surface 38 to the outer surface 36 is about 0.010 inch. The diameter of lumen 39 is substantially uniform through most of catheter 22 in the illustrated embodiment, and depends on the outer diameter of the obturator 24 (or the outer diameter of obturator 24 is chosen based on the diameter of lumen 39). It is estimated that particularly useful diameters of lumen 39 range from about 0.05 inch to about 0.2 inch. Of course, there may be applications that require larger or smaller dimensions for catheter 22. The catheter 22 can be curved or straight as may be desired or necessary for a particular medical procedure.

In some embodiments, the catheter 22 is made of or includes a biocompatible radiopaque material, so as to give the physician the option to visualize catheter 22 by fluoroscopy or X-rays. For example, catheter 22 can be made of silicone, polyurethane, or any other biocompatible material in which barium sulfate or another radiopaque material is mixed or suspended. As another example, distal end 34 of catheter 22 may be configured to include a guidance element for visualizing, guiding and/or positioning the rotational orientation of catheter 22 within the vasculature of a patient. Such guidance elements include one or more markers, sensors, and/or emitters. For instance, the distal end 34 and/or other part(s) of catheter 22 may include a radiopaque marker (e.g. a bead of biocompatible metal) to permit visualization or other location of such part(s), in particular their position and/or orientation within a patient's body.

The illustrated embodiment of catheter 22 includes a single central longitudinal lumen. It will be understood that other embodiments may have dual lumens (side-by-side, one within another or coaxial) or multiple such lumens. In embodiments with multiple or dual lumens, the obturator 24 is inserted in only one of the lumens, so as to preserve the integrity of the other lumen(s).

Echogenic obturator 24 includes a shaft 40 that spans from a proximal end 42 to an opposite distal end 44. Obturator 24 aids in positioning the catheter 22 in a desired location and orientation within the vasculature of a patient. Proximal end 42 in the illustrated embodiment includes a handle 46. In one form, the handle 46 is a controller for advancing and/or retracting the obturator 24 with respect to catheter 22, as well as for advancing and/or retracting the catheter 22 and obturator 24 attached together. The illustrated embodiment of handle 46 includes a cap 48 that is sized and configured to engage and retain the hub 26. Cap 48 defines a central aperture 50 defining an outer flange 51 that is internally threaded with threads 52. As will be explained further below, flange 51 and threads 52 are sized and configured to threadedly connect with threads 72 on the proximal portion 70 of hub 26. Cap 48 is configured to attach to a proximal portion 70 of hub 26 in a manner such that the cap 48 is easily disconnected from the hub 26. Obturator 24 is a single piece in this embodiment, with shaft 40 ending within aperture 50 and substantially in the center of aperture 50.

In another embodiment illustrated in FIG. 2, cap 148 is similar in most respects to cap 48. Cap 148 forms a non-threaded interference fit or friction fit connection with a proximal flange portion 170 of the hub 26, which is similar in most respects to portion 70 of hub 26. Additionally, a distal hub portion 180 is configured to form an interference or friction fit connection with a proximal end 132 of catheter 122. As another example (not illustrated), the cap may be configured to form a bayonet-type connection with the proximal portion of the hub. Typically, the bayonet connection consists of a male side with one or more pins positioned on the proximal portion of the hub, and a female receptor with matching L slots (and may include one or more springs) positioned in the cap wherein the connection between the pins and slots keep the cap and hub locked together.

Shaft 40 has an external diameter which is approximately the same or slightly smaller in size than the diameter of lumen 39 of catheter 22, so that shaft 40 can be relatively easily moved within catheter 22. Minimizing the tolerance between shaft 40 and lumen 39 permits a smaller overall catheter 20 with better support for catheter 22. In the illustrated embodiment, the obturator 24 has a generally solid uniform cross section of the same shape as lumen 39, and includes a lumen 53 extending from a proximal opening 54 at proximal end 42 to a distal opening 55 at the distal end 44. Lumen 53 is small compared to the overall diameter of shaft 40 and is sized for passage of a guide wire 56. As indicated above, guide wire 56 can be used for placement or withdrawal of catheter 20, by moving catheter 20 (e.g. catheter 22 with attached obturator 24) over guide wire 56, so that wire 56 moves through lumen 53 and openings 54, 55. In particular embodiments, the guide wire 56 is made of a biocompatible metal, which has good reflective characteristics with respect to ultrasound, or other echogenic material(s). Such embodiments of guide wire 56 may aid the physician in placing the catheter 20 under ultrasound visualization. Other embodiments of catheter 20 or obturator 24 may not include a guide wire 56, particularly if the length of travel through the vasculature is minimal. Shaft 40 can have a solid or hollow cross-section, e.g. with or without a lumen as described, and the cross-section of shaft 40 may be uniform. Shaft 40 can also be flexible or rigid. Beneficially, a flexible shaft 40 will further aid the physician in placing the shaft 40 and catheter 22 within the turns of the vasculature of a patient.

All or a portion of obturator 24 (e.g. part or all of shaft 40 or its distal end 44) is made of an echogenic material that is highly visible under sonography, ultrasound, or other ultrasonic methods. As such, X-rays or fluoroscopy are not required to place the catheter 20 in the vasculature of the patient. Some types of echogenic material include foamed plastic and/or plastic imbedded with ultrasonically reflective particles. Some examples of ultrasonically reflective particles include zinc oxide, iron oxide, titanium dioxide, platinum oxide, and silver oxide. However, other types of echogenic material can be used for obturator 24. In addition, or as another configuration for obturator 24, shaft 40 can include a plurality of discrete echogenic markers placed or positioned along the length of the shaft 40 (e.g. along the outer surface of shaft 40) at one or more desired intervals. In one such example, an echogenic marker is positioned at the distal end 44 of shaft 40, and additional echogenic markers are positioned at intervals of about 5.0 cm proximally along the length of the shaft 40. By observing the passage of such marker(s) with respect to a substantially stationary part of an ultrasound image (e.g. an anatomic part such as a bone or a previously-inserted device), the physician knows how far the marker(s), and thus obturator 24 and catheter 22, have traveled within the body. Knowing how far to insert catheter 22 from the type of medical procedure to be performed and the patient's vasculature, the physician can assure at least approximately proper placement by counting the number of markers that pass a given location (e.g. the entrance to the body or a particular location in a vessel), to estimate how much of catheter 22 has been inserted past that location, or by observing a marker on end 44 of shaft 40 approaching a desired location for the distal end 34 of catheter 22.

As previously noted, the guide wire 56 can also be made of metal or other echogenic material. Echogenic markers as discussed above may be placed in particular locations with respect to either an echogenic or non-echogenic guide wire 56, such as at defined or predetermined intervals along the wire to assist the physician in visualizing where obturator 24 and/or catheter tube 22 are with respect to guide wire 56, or how far obturator 24 and/or catheter 22 have traveled with respect to guide wire 56.

In the illustrated embodiment, the adapter or hub 26 includes a proximal flange portion 70 opposite a distal body portion 80. Flange portion 70 is adapted to connect securely to cap 48 of obturator 24, while body portion 80 is adapted to connect securely to catheter 22. Particular embodiments of portions 70 and 80 are circular in cross-sectional shape, for ease in connection to obturator 24 and catheter 22. However, it will be understood that portions 70 and 80 can have other cross-sectional configurations such as square, oval, octagonal, or other shape that may be compatible with secure fitting to catheter 22 and/or obturator 24.

Flange portion 70 has a larger diameter in this embodiment than that of body portion 80. As previously noted, flange portion 70 is configured to be secured within cap 48 of obturator 24, so as to releasably engage and retain cap 48. For example, in the illustrated embodiment flange portion 70 includes external threads 72 which threadedly engage with internal threads 52 of flange 51 of cap 48. The physician can detach the connection between the hub 26 and the cap 48 by rotating the cap 48 around the hub 26 (e.g. flange portion 70) to unscrew the cap 48 from the hub 26, and if needed can attach (or reattach) cap 48 to hub 26 by screwing cap 48 onto hub 26. As described previously, proximal flange portion 70 and cap 48 can be configured differently to releasably engage and retain one another in other embodiments.

Distal body portion 80 of hub 26 is configured to securely engage and maintain proximal end 32 of catheter 22 around it. In the illustrated form, body portion 80 includes a plurality of teeth or barbs 82 that engage and bite into or otherwise hold proximal end 32 of catheter 22. As will be appreciated, there are many different types of configurations that can be used to retain proximal end 32 of catheter 22 on distal hub portion 80. In other embodiments, for example, body portion 80 forms an interference or press-fit connection with the proximal end 32 of catheter 22. As another example, such teeth 82 of body portion 80 can take the form of continuous or interrupted threads that engage the inside of proximal end 32 of catheter 22. Such threads may engage a smooth, flexible wall of catheter 22, or if proximal end 32 has been tapped, such thread may thread into threads within proximal end 32.

Hub 26 further includes a channel 84 extending through both portions 70 and 80. In the illustrated embodiment, channel 84 is linear, and sized and configured to accept passage of shaft 40 of obturator 24. In particular embodiments in which proximal end 32 of catheter 22 is flared outward, the diameter of channel 84 may be approximately the same as the diameter of lumen 39 of catheter 22. In such embodiments, shaft 40 of obturator 24 passes just as well during insertion, retraction, rotation or other movement with respect to hub 26 as it does with respect to catheter 22.

Hub 26 can be made of any suitable material that is compatible with the echogenic obturator 24 and the catheter 22. Hub 26 may be of a biocompatible material similar or identical to one or both of catheter 22 and obturator 24 for ease of manufacture and use, but need not be biocompatible since hub 26 remains outside during use of catheter 20. Body portion 80 of hub 26 is inserted in the proximal end 32 of catheter 22 and the obturator 24 is inserted through hub 26 and into catheter 22 such that the cap 48 retains the proximal hub portion 80. Hub 26 consequently functions to maintain the position of the obturator 24 relative to the catheter 22. As previously noted, the distal end 44 of the obturator 24 may be aligned or flush with the distal end 34 of the catheter 22 while hub 26 operates to couple the obturator 24 to the catheter 22.

As shown in FIG. 1, body portion 80 of hub 26 forms a first connection with the proximal end 32 of catheter 22, and the flange portion 70 forms a second connection with the cap 48 of the obturator 24. This two-connection configuration is beneficial to the physician or other user in that the obturator 24 is easily removed from the catheter 22 by twisting the obturator 24 about the hub 26 to unscrew the cap 48 from the proximal hub portion 70, while hub 26 stays firmly engaged to catheter 22. The user is not required to hold the catheter 22 in its placement or final position within the patient during removal of obturator 24, since the obturator 24 is not directly connected to catheter 22. Such easy separation and removal of obturator 24 from hub 26 with minimal disturbance to catheter 22 is convenient for the physician and generally less traumatic for the patient.

In other embodiments, hub 26 may be replaced by or take the form of a luer connector inserted and securely connected within the proximal end 32 of catheter 22, with cap 48 of obturator 24 configured to attach to the luer connector. In this form, the cap and the luer connector would be configured to be securely engaged to each other, while be releasable through operation of the luer connector mechanism. In this form, to remove the obturator from the catheter, the physician simply rotates the cap 48 around the luer connector and pulls the obturator 24 away from the luer connector and out of catheter 22.

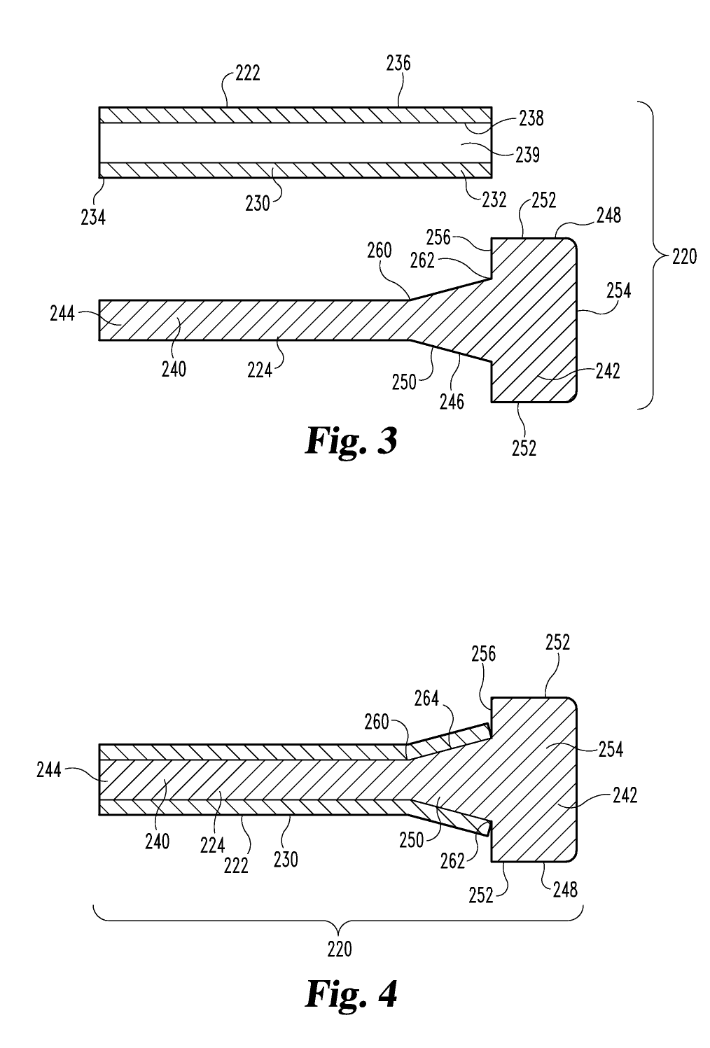

In other embodiments, cap 48 and hub 26 are a single piece that is configured to securely attach within proximal end 32 of catheter 22. One embodiment of a single piece consisting of the cap and hub is described below and illustrated in FIGS. 3 and 4. In another configuration, the echogenic obturator 24 includes a plurality of teeth that engage and retain the proximal end 32 of catheter 22. In either embodiment, after positioning the catheter 22 in the patient, the physician or other technician is required to maintain the catheter 22 in its proper position while removing the obturator 24 or combined hub 26 and obturator 24 from the catheter 22 so as not to disturb the orientation of catheter 22 in the patient.

In some embodiments, the catheter 22 will remain within a patient's body for an extended period of time. During such catheterizations, drugs or other therapeutic or diagnostic substances or devices can be delivered into the patient's blood stream or to a specific body location (e.g. a vascular location) through the catheter 22. To accomplish such delivery, after implantation of the catheter 20 in the patient as noted above, the physician removes the obturator 24 from the implanted catheter 22 by detaching cap 48 from hub 26. As noted above, such detaching depends on the connection(s) between cap 48 and hub 26, and may involve unscrewing cap 48 from flange portion 70 of hub 26, or disengaging an interference or press-fit between cap 48 and flange portion 70 of hub 26.

With cap 48 and hub 26 disengaged, the user can pull obturator 24 through lumen 39 of catheter 22 and channel 84 of hub 26, and discard or set aside obturator 24. The user determines an appropriate length of catheter 22 to leave extending from the patient, e.g. between none or a very nominal length and 1 meter, and may determine an attachment position on catheter 22 for a device such as an infusion port (not shown) or other mechanism suited to the treatment being given. Next the user trims catheter 22 to the desired length or to the desired position for a port or other device. Such trimming occurs distal of proximal end 32, and thus removes end 32 (with its flared portion, if present) and hub 26 inserted therein. If desired, hub 26 can be separately removed from end 32 prior to or after the trimming and retained.

As noted, an infusion port or other attachment for enabling introduction of substances or devices into catheter 22 can be attached to the trimmed catheter 22, for example to the new proximal end 32 created by the trimming. If necessary, the infusion port or other attachment may be removed while the catheter 22 remains implanted in the patient's body, so that catheter 22 can be used for other purposes or with new attachment(s). Devices or attachments other than ports that can be attached to catheter 22 include pumps, delivery devices, subcutaneously implanted reservoirs, injection ports, or other therapeutic or diagnostic devices related to introduction and/or removal of fluid or devices from the body.

As a particular example, echogenic catheter 20 may be inserted into a blood vessel over a guide wire 56, for example, using the now well-known Seldinger percutaneous entry technique. Referring to FIG. 1, catheter 22 is assembled with obturator 24 and hub 26 as described. Distal body portion 80 of hub 26 is inserted in the proximal end 32 of the catheter 22 (as by pressing or rotating/threading) to secure the proximal end 32 of the catheter 22 on the body portion 80. The distal end 44 of the obturator 24 is inserted and advanced through channel 84 in hub 26 and lumen 39 in catheter 22 until it is adjacent or substantially aligned with end 34 of catheter 22. The cap 48 of obturator 24 is releasably connected to flange portion 70, as by threading or pressing on. Obturator 24 is thus securely engaged to hub 26, and hub 26 securely engaged to catheter 22, with end 44 of obturator 24 and end 34 of catheter 22 at or near each other. This configuration enables the physician to control catheter 22 using obturator 24 (e.g. its cap 48) and to accurately place the distal end 34 of catheter 22 by visualizing the position of the echogenic distal end 44 of obturator 24 during the insertion, as described below.

With catheter 22 thusly assembled with obturator 24 and hub 26, an introducer needle is inserted through the skin and/or other tissue and into the patient's vasculature, e.g. a peripheral vein. A guide wire 56 is inserted through the needle and into the vessel to a distance desired by the physician. The needle is removed and an introducer that consists of an appropriately sized dilator and an outer sheath is inserted over the guide wire 56 and into the patient. The tissue surrounding the guide wire 56 is dilated with the dilator. The dilator and the guide wire 56 are then removed, leaving the outer sheath in place. Catheter 20 is then inserted into the patient through the outer sheath. The physician feeds catheter 20 (catheter 22 with obturator 24 and hub 26) into the outer sheath so that distal end 34 of catheter 22 is in a desired position within the vessel. The outer sheath and obturator 24 are then removed.

As previously discussed, to aid in positioning the catheter 22, at least part of obturator 24 (e.g. its distal end 44) is made of or includes an echogenic material, making it highly visible by ultrasound or sonography. Obturator 24 may include a plurality of echogenic markers positioned at a designated interval along the shaft 40 as described above. Alternatively or additionally, as described previously, guide wire 56 can be made of an echogenic material or include a plurality of echogenic markers positioned along the length of the guide wire 56. As catheter 20 is advanced along guide wire 56, the physician can observe via ultrasound the progress of catheter 20 relative to the guide wire 56 and to viewable anatomical structures. When the distal end 44 of obturator 24 is observed on the ultrasound monitor to be at a desired anatomical location, or at the distal end of guide wire 56 if that distal end has been placed at the desired location, then the physician knows that end 34 of catheter 22 (which is aligned with or adjacent to end 44 of obturator 24) is at the desired location.

When the desired position of the distal end 34 of the catheter 22 within the patient is achieved, the physician unlocks the cap 48 from the hub 26, as by rotating cap 48 about hub 26 to unscrew or release a bayonet connection, or by pulling apart or otherwise releasing an interference fit between cap 48 and flange portion 70 of hub 26. The guide wire 56 is removed by pulling out through lumen 53 of obturator 24, and obturator 24 is removed by pulling cap 48 to move shaft 40 out through lumen 39 of catheter 22 and channel 84 of hub 26. In other embodiments and techniques for positioning catheter 22, guide wire 56 and obturator 24 may be removed simultaneously, i.e., without pulling wire 56 through lumen 53 of obturator 24.

With wire 56 and obturator 24 removed, catheter 22 and hub 26 remain. The distal end 34 of catheter 22 remains in its intended position (e.g. within the patient's vessel at the desired location) and hub 26 is attached to the proximal end 32. In many circumstances when a physician intends to attach and use an infusion port, significant blood loss through an inserted catheter (here, catheter 22) is usually not a concern, and therefore the physician may not need to take steps to stop blood flow through catheter 22 immediately following withdrawal of wire 56 and obturator 24. Of course, the physician may desire to clamp catheter 22 outside the patient's body or take other steps to ensure little or no blood flow through catheter 22 after withdrawing obturator 24.

The physician may trim catheter 22 to a desired length that extends outside of the patient or to a desired length that remains implanted under a patient's skin surface, thereby cutting off proximal end 32 with hub 26, and discard that cut-off catheter portion. Now the trimmed end (a new proximal end 32) of the catheter 22 is free for attachment to an infusion port or other medical device and the distal end 34 of the catheter 22 remains properly positioned within the vessel. While the structures and methods described above seek to reduce or eliminate X-ray radiation doses in catheter placement, it is understood that if determination of the positioning and/or orientation of distal end 34 or other parts of catheter 22 becomes necessary at a later time, fluoroscopy or X-rays can be used particularly if catheter 22 is made of or includes radiopaque material, and/or includes radiopaque markers, sensors, and/or emitters.

As described above, catheter 20 includes a catheter 22, obturator 24 and hub 26 assembled together. Assembly may take place prior to use by a physician or other professional. For example, following individual manufacture of each of catheter 22, obturator 24 and hub 26, the parts can be assembled together, sterilized, and packaged for the physician's use. The physician need only open the package to have a properly assembled catheter 20 available, to be inserted over a placed guide wire or inserted into an introducer sheath as discussed above. In other embodiments, sterilized parts may be provided to the physician or technician for assembly at the time of use.

FIGS. 3 and 4 illustrate another embodiment of a delivery catheter assembly or echogenic catheter assembly 220 of the present disclosure. Assembly 220 is similar to assembly 20, and so for the sake of brevity similar features will not be discussed again. Assembly 220 includes a hollow catheter 222 and an echogenic obturator 224. As described in more detail below, the echogenic obturator 224 is inserted in the catheter 222 and thereafter the echogenic obturator 224 retains the catheter 222 until the physician desires to remove the obturator 224.

Catheter 222 is similar to catheter 22. Catheter 222 includes an elongate flexible body 230 having a proximal end 232 and an opposite distal end 234. As illustrated in FIG. 3, body 230 has a substantially uniform diameter from the proximal end 232 to the distal end 234. In an alternate embodiment, body 230 may be similar to body 30 wherein the proximal end 232 flares radially outwardly as described above. In either embodiment, a hub portion 250 of the echogenic obturator 224 forms an interference or press fit connection with proximal end 232.

The hollow catheter 222 defines an outer surface 236 and an inner surface 238 surrounding a lumen 239. Lumen 239 has a diameter sized to receive the obturator 224 and is substantially uniform through the length of catheter 222 and depends on the outer diameter of the obturator 224 (or the outer diameter of obturator 224 is chosen based on the diameter of lumen 239).

Echogenic obturator 224 includes a shaft 240 that spans from a proximal end 242 to an opposite distal end 244. Proximal end 242 in the illustrated embodiment is configured differently than proximal end 42 as illustrated in FIG. 1. Proximal end 242 includes a cap portion 248 and a hub portion 250. As discussed previously, the cap portion 248 and the hub portion 250 are one embodiment or example of a single piece that is configured to securely attach within proximal end 232 of catheter 222.

Cap portion 248 has a rectangular cross-sectional shape in the illustrated embodiment. In other embodiments the cap portion 248 may be configured differently such as circular, trapezoidal, or an ergonomic shape to comfortably fit in the surgeon's hand. The cap portion 248 has a solid cross-sectional shape. In other embodiments the cap portion 248 may be hollow or include a cut-out portion. For example, cap portion 248 may include a plurality of holes sized to receive the fingers of the surgeon to aid the surgeon in grasping the cap portion 248. In the illustrated embodiment, cap portion 248 includes a pair of longitudinal sides 252, a first lateral side 254, and a second lateral side 256 wherein the first and second lateral sides 254 and 256 are substantially perpendicular to the pair of longitudinal sides 252. The second lateral side 256 has a length that is longer than the width of the hub portion 250 as described below. The longer length of second lateral side 256 is beneficial in that the second lateral side 256 functions as an end stop to limit the rearward movement of the proximal end 232 of the catheter 222 when the catheter 222 is assembled with the obturator 224.

Hub portion 250 is sized and configured to securely engage and maintain proximal end 232 of catheter 222 around it. The hub portion 250 is a conical shape with a first diameter 260 that widens to a second diameter 262 with a substantially straight surface 264 that spans between the diameters 260 and 262. The first diameter 260 is substantially the same size diameter as the shaft 240. The second diameter 262 intersects the second lateral side 256, with the second diameter 262 being smaller than the diameter or dimension second lateral side 256 in this embodiment. In other embodiments, the hub portion 250 may be shaped differently. For example, the surface 264 may curve or bow outwardly from the shaft 240. In yet in other embodiments, the surface 264 may include a plurality of teeth or other mechanism to engage or bite into and retain the proximal end 232 of the catheter 222.

Hub portion 250 functions to retain the catheter 222 on the obturator 224. In an assembled state illustrated in FIG. 4, the distal end 244 of the obturator 224 is aligned or flush with the distal end 234 of the catheter 222.

Shaft 240 is similar to shaft 40, and so similar details will not be discussed. Unlike obturator 24, obturator 224 does not include a lumen. Instead, obturator 224 has a solid cross-sectional shape. In other embodiments, obturator 224 could include a lumen similar to lumen 53 of obturator 24 to receive a guide wire similar to guide wire 56.

Similar to obturator 24, all or a portion of obturator 224 (e.g. part or all of shaft 240 or its distal end 244) is made of an echogenic material that is highly visible under sonography, ultrasound, or other ultrasonic methods. Similar types of echogenic material as described above can be used for both obturators 24 and 224.

The echogenic catheter 220 may be inserted in a blood vessel as described next. Referring to FIGS. 3 and 4, catheter 222 is assembled with the obturator 224. First, the distal end 244 of the obturator 224 is inserted into the lumen 239 at the proximal end 232 of the catheter 222. The obturator 224 is advanced in the lumen 239 of the catheter 222 until the distal end 244 of the obturator 224 is adjacent or substantially aligned with distal end 234 of catheter 222. The proximal end 232 of the catheter 222 is pulled over the hub portion 250 of the obturator 224 until the proximal end 232 of the catheter 222 engages the second lateral side 256. The hub portion 250 securely retains the proximal end 232 of the catheter 222 by an interference type connection wherein the proximal end 232 is stretched slightly or deformed as the proximal end 232 is pulled onto the hub portion 250. The obturator 224 is securely engaged to the catheter 222 with distal end 244 of obturator 224 and distal end 234 of catheter 222 at or near each other. As described previously, this configuration enables the physician to control catheter 222 by visualizing the position of the echogenic distal end 244 of obturator 224 during the insertion.

With catheter 222 thusly assembled with obturator 224, an introducer needle is inserted through the skin and/or other tissue and into the patient's vasculature, e.g. a peripheral vein. A guide wire is inserted through the needle and into the vessel to a distance desired by the physician. The needle is removed and an introducer that consists of an appropriately sized dilator and an outer sheath is inserted over the guide wire and into the patient. The tissue surrounding the guide wire is dilated with the dilator. The dilator and the guide wire are then removed, leaving the outer sheath in place. Catheter 220 is then inserted into the patient through the outer sheath. The physician feeds catheter 220 (catheter 222 with obturator 224) into the outer sheath so that distal end 234 of catheter 222 is in a desired position within the vessel. The outer sheath and obturator 224 are then removed.

When the desired position of the distal end 234 of the catheter 222 within the patient is achieved, the physician pulls cap portion 248 from catheter 222 or rotates cap portion 248 about catheter 222 to release the interference fit between hub portion 250 and proximal end 232 of catheter 222. The obturator 224 is removed by pulling cap portion 248 to move shaft 240 out through lumen 239 of catheter 222. While the cap portion 248 is being pulled, the medical practitioner should also grasp a portion of the catheter 222 extending outside or exteriorly of the patient's body so that the distal end 234 of the catheter 222 remains in its intended and desired position.

With obturator 224 removed, the distal end 234 of catheter 222 remains in its intended position (e.g. within the patient's vessel at the desired location) and the proximal end 232 of the catheter 222 is available for trimming. As described above, the physician may trim the proximal end 232 of catheter 222 to a desired length and attach an appropriate medical device or infusion port.

While the disclosure has been illustrated and described in detail in the drawings and foregoing description, the same is to be considered as illustrative and not restrictive in character, it being understood that only preferred embodiments have been shown and described and that all changes, equivalents, and modifications that come within the spirit of the disclosures defined by following claims are desired to be protected. Particular features described with respect to one embodiment or structure are usable with other embodiments or structures disclosed herein. All publications, patents, and patent applications cited in this specification are herein incorporated by reference as if each individual publication, patent, or patent application were specifically and individually indicated to be incorporated by reference and set forth in its entirety herein.

1. A method for positioning a catheter within vasculature of a patient, the method comprising the steps of:
providing a catheter with an echogenic obturator installed therein and a hub positioned in the catheter to securely engage the catheter, the catheter having a distal end opposite a proximal end, the echogenic obturator having a distal end opposite a proximal end, wherein the distal ends of the catheter and the echogenic obturator are substantially aligned, and the proximal end of the echogenic obturator releasably retains the hub;
inserting the catheter and the echogenic obturator into the vasculature of the patient;
determining the position of the distal end of the catheter within the vasculature of the patient with ultrasound imaging of the distal end of the echogenic obturator; and
releasing the echogenic obturator from the hub after the determination of the position of the distal end of the catheter.

2. The method of claim 1, wherein the proximal end of the echogenic obturator includes a cap configured to retain the hub.

3. The method of claim 2, wherein the cap includes internal threads and the hub includes a proximal flange portion opposite a distal body portion, the proximal flange portion having external threads configured to threadedly engage the threads of the cap.

4. The method of claim 3, wherein the distal body portion includes a plurality of teeth configured to securely engage the interior of the proximal end of the catheter.

5. The method of claim 3, wherein releasing the echogenic obturator includes rotating the echogenic obturator around the hub to unscrew the threads of the cap from the threads on the proximal hub portion.

6. The method of claim 1, further comprising:
removing the echogenic obturator from the catheter and the hub.

7. A delivery catheter for insertion into vasculature of a patient, the delivery catheter comprising:
a catheter having a distal end opposite a proximal end;
an obturator having a distal end opposite a proximal end, the obturator disposed at least partially within the catheter wherein the distal ends of the catheter and the obturator are substantially aligned, the distal end of the obturator made of an echogenic material that is visible with sonography or ultrasound imaging; and
a hub having a proximal flange portion opposite a distal body portion, the distal body portion configured to securely engage the interior of the catheter, the proximal flange portion configured to releasably connect with the proximal end of the obturator.

8. The delivery catheter of claim 7, wherein the proximal end of the obturator includes a cap, each of the cap and the proximal flange portion being threaded so as to releasably connect with one another to retain the obturator on the hub.

9. The delivery catheter of claim 7, wherein the distal body portion includes a plurality of teeth configured to grip the interior of the proximal end of the catheter.

10. The delivery catheter of claim 7, wherein the distal end of the catheter has a first diameter and the proximal end of the catheter is flared radially outwardly from the first diameter to a second diameter that is larger than the first diameter, the proximal end of the catheter being configured to receive the distal body portion of the hub.

11. The delivery catheter of claim 7, wherein the obturator includes a guide wire that is made of echogenic material.

12. The delivery catheter of claim 7, wherein the obturator includes a shaft that spans from the obturator distal end to the obturator proximal end, and the obturator includes a plurality of markers made of echogenic material, each of the plurality of markers positioned at a substantially uniform interval along the shaft.

13. The delivery catheter of claim 12, wherein the interval is about 5 cm.

14. The delivery catheter of claim 12, wherein one of the plurality of markers is positioned on the distal end of the obturator.

15. The delivery catheter of claim 7, wherein the catheter includes a radiopaque material to enable visualization of the catheter within the vasculature of the patient by fluoroscopy or X-rays.

16. A method of making a delivery catheter, comprising:
providing a catheter having a distal end opposite a proximal end, an obturator having a distal end opposite a proximal end, the distal end of the obturator made of an echogenic material, and a hub having a distal body portion opposite a proximal flange portion;
inserting the distal body portion of the hub into the proximal end of the catheter to securely engage the catheter thereon;
inserting the distal end of the obturator into the proximal flange portion of the hub and the catheter until the distal end of the obturator is substantially aligned with the distal end of the catheter; and
attaching the proximal end of the obturator to the proximal flange portion of the hub.

17. The method of claim 16, wherein the attaching the proximal end of the obturator includes forming a releasable connection between the proximal end of the obturator and the proximal flange portion of the hub.

18. The method of claim 17, wherein the proximal end of the obturator includes a cap, the cap and the proximal flange portion each being threaded and sized to threadedly engage one another when the obturator is assembled with the hub.

19. A delivery catheter for insertion into vasculature of a patient, the delivery catheter comprising:
a catheter having a distal end opposite a proximal end; and
an obturator having a distal end opposite a proximal end, the obturator disposed at least partially within the catheter wherein the distal ends of the catheter and the obturator are substantially aligned, the distal end of the obturator made of an echogenic material that is visible with sonography or ultrasound imaging, the proximal end of the obturator configured to releasably connect with the proximal end of the catheter.

20. The delivery catheter of claim 19, wherein the proximal end of the obturator includes a hub portion having a first diameter and a second diameter, the hub portion is flared radially outwardly from the first diameter to the second diameter that is larger than the first diameter, the hub portion being configured to receive the proximal end of the catheter.
